(19) United States
(12) Patent Application Publication
(10) Pub. No.: US 2025/0064639 A1
(43) Pub. Date: Feb. 27, 2025

(54) INTRA-OPERATIVE VISUALIZATION, MEASUREMENT, AND ASSISTANCE FOR OPHTHALMIC TREATMENTS

(71) Applicant: Alcon Inc., Fribourg (CH)

(72) Inventors: Patrick TERRY, Goleta, CA (US); Rafael CHAN, Fort Worth, TX (US); Regi KUHN, Fort Worth, TX (US); Satish YALAMANCHILI, Irvine, CA (US); Aaron MAYS, Fort Worth, TX (US); Nanhong LOU, Southlake, TX (US)

(21) Appl. No.: 18/812,792

(22) Filed: Aug. 22, 2024

Related U.S. Application Data
(60) Provisional application No. 63/578,360, filed on Aug. 23, 2023.

(51) Int. Cl. A61F 9/007; A61B 3/10; A61B 3/16; A61B 34/20; A61B 90/25; A61F 2/16

(52) U.S. Cl. CPC A61F 9/00781; A61F 2/1662; A61F 9/00745; A61B 3/102; A61B 3/16; A61B 2034/2065; A61B 90/25

(57) ABSTRACT

A system for performing ophthalmic treatments includes a surgical microscope configured to capture surface images of an eye of a patient and an imaging device mounted to the surgical microscope and configured to capture section images of the eye of the patient. A controller is coupled to the surgical microscope and the imaging device, the controller configured to receive the surface images and section images and provide feedback facilitating performance of the ophthalmic treatments based on the section images and the surface images. Feedback may relate to avoiding bag rupture during phacoemulsification, safely performing retinal membrane peeling, placing an IOL, treating glaucoma, or performing other ophthalmic treatments.

### CROSS-REFERENCE TO RELATED APPLICATIONS

This application claims priority to U.S. Provisional Application No. 63/578,360, filed on Aug. 23, 2023, which is hereby incorporated by reference in its entirety.

### BACKGROUND

The present disclosure relates generally to performing ophthalmic surgery.

Light received by the eye is focused by the cornea and lens of the eye onto the retina at the back of the eye, which includes the light sensitive cells. The area between the cornea and the lens is known as the anterior segment. The interior of the eye between the lens and the retina is known as the posterior segment and is filled with a transparent gel known as the vitreous. Many ocular pathologies may be treated by performing ophthalmic treatments in the interior or posterior segments.

It would be an advancement in the art to facilitate the performance of ophthalmic treatments.

### SUMMARY

In certain embodiments, a system for performing ophthalmic treatments includes a surgical microscope configured to capture surface images of an eye of a patient and an imaging device mounted to the surgical microscope and configured to capture section images of the eye of the patient. A controller is coupled to the surgical microscope and the imaging device, the controller configured to receive the surface images and section images and provide feedback facilitating performance of the ophthalmic treatments based on the section images and the surface images.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a surgical microscope for use by a surgeon performing ophthalmic treatments.

FIG. 1B illustrates a surgical microscope with an accessory imaging device mounted thereto in accordance with an embodiment of the present invention.

FIG. 1C illustrates the accessory imaging device used in combination with a gonioscope in accordance with certain embodiments.

FIG. 1D illustrates the accessory imaging device that is adjustable with respect to the surgical microscope in accordance with certain embodiments.

FIG. 2 illustrates an eye undergoing treatment for glaucoma in accordance with certain embodiments.

FIG. 3 is a process flow diagram of a method for providing intra-operative assistance during a glaucoma treatment in accordance with certain embodiments.

FIGS. 4A and 4B illustrate examples of visual assistance displayed during a glaucoma treatment in accordance with certain embodiments.

FIG. 5 illustrates a retina undergoing a membrane peeling treatment with visual assistance displayed in accordance with certain embodiments.

FIGS. 6A to 6C are process flow diagrams of methods for providing visual assistance during a membrane peeling treatment in accordance with certain embodiments.

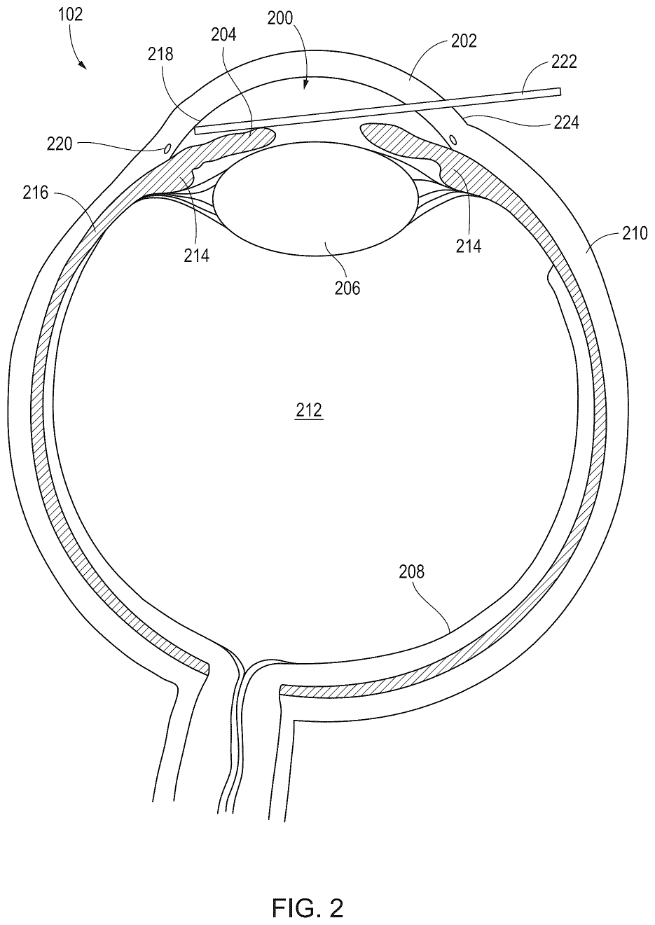

FIG. 2A is a diagram showing an ophthalmic treatment performed using the surgical instrument and imaging device in accordance with certain embodiments.

FIGS. 7A and 7B illustrate a cataract treatment including phacoemulsification and placement of an intraocular lens.

FIGS. 8A to 8F are process flow diagrams of methods for providing assistance during a cataract treatment in accordance with certain embodiments.

### DETAILED DESCRIPTION

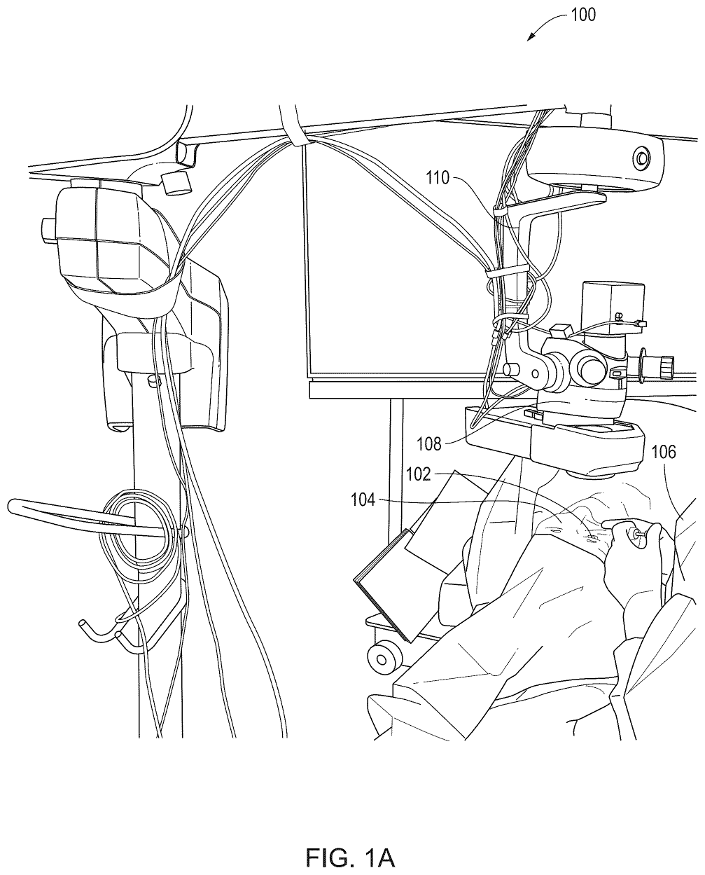

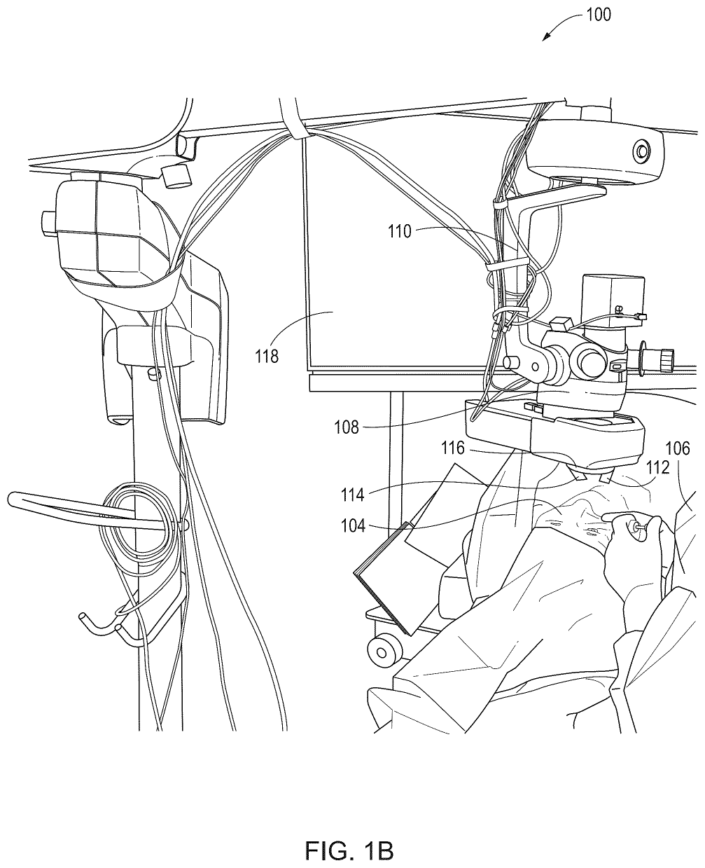

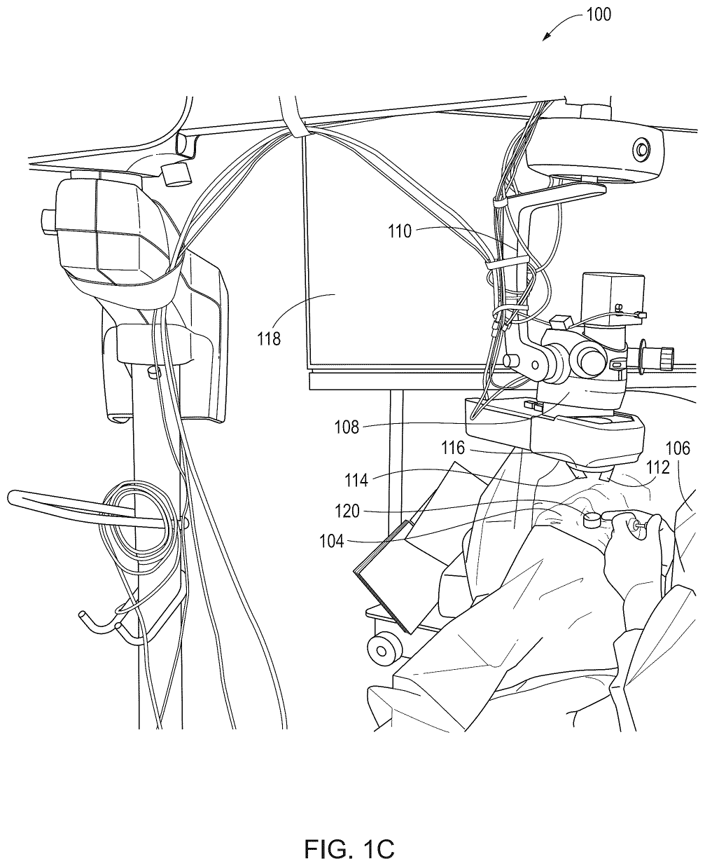

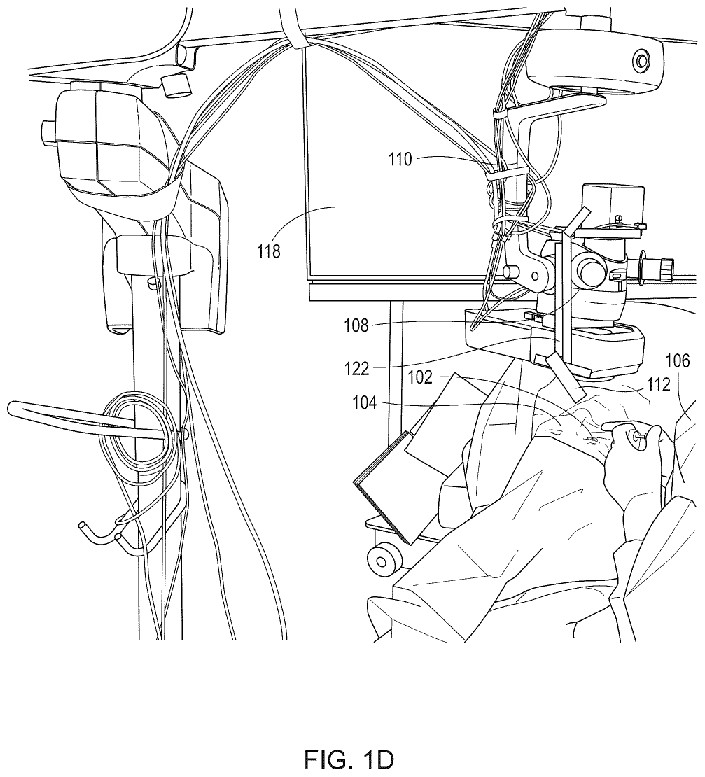

Referring to FIG. 1A, an operating environment 100 may be used to perform an ophthalmic treatment on an eye 102 of a patient 104 by a surgeon 106. The operating environment 100 may include a surgical microscope 108 suspended from a support 110 facilitating positioning of the surgical microscope 108 over the eye 102 at a height desired by the surgeon 106. For example, surgical microscope 108 may be implemented as the NGENUITY 3D VISUALIZATION SYSTEM provided by Alcon Inc. of Fort Worth Texas.

Referring to FIG. 1B, an imaging device 112 mounts to the surgical microscope 108 and provides imaging according to one or both of (a) a different imaging modality than the surgical microscope 108 (b) a different point of view or level of zoom than the surgical microscope 108, such as visible light camera having wider viewing angle than the surgical microscope 108. One or more additional accessory devices 114 may also mount to the surgical microscope 108 to facilitate performance of an ophthalmic treatment. The accessory device 114 may be another imaging device according to the same or different imaging modality as the imaging device 112. The accessory device 114 may also be a sensor other than imaging device, such as an intraocular pressure (IOP) sensor (e.g., contact or non-contact tonometry (NCT) IOP sensor) or other type of sensor. The accessory device 114 may also be a light source for illuminating the eye 102. Examples of imaging modalities used to implement the surgical microscope 108, imaging device 112, and accessory device 114 may include a visible light camera, an infrared light camera, a fundus autofluorescence (FAF) camera, a multispectral imaging (MSI), a hyperspectral imaging (HSI) camera, wide angle viewing (WAV) camera, optical coherence tomography (OCT) imaging device, or a scanning laser ophthalmoscope (SLO). The accessory device 114 may be embodied as a laser or sonic range finder or imaging device.

In the illustrated embodiment, a mounting ring 116 secures to the surgical microscope 108, such as around an objective lens of the surgical microscope and/or optical axis of the surgical microscope 108. The imaging device 112 and accessory device 114 secure to the mounting ring 116 either on opposite sides of the ring 116, e.g., 180 degrees offset from one another around the center of the ring 116, or at some other position. The mounting ring may provide mounting points to which any of the imaging devices 112 and accessory devices 114 listed above may removably mount, including intraoperatively swapping one imaging device and/or accessory device 114 for a different imaging device 112 and/or accessory device 114.

Images received from or derived from images received from the surgical microscope 108, the imaging device, and possibly the accessory device 114 may be displayed in either (a) a display device (e.g., a stereoscopic display device) within the surgical microscope 108 or (b) an external display device 118, such as a monitor, projector, or other display device.

Referring to FIG. 1C, in some embodiments, the surgical microscope 108 may be used in combination with a gonioscope 120, such as when performing ophthalmic treatments for glaucoma. The gonioscope 120 may be implemented as a gonio prism or gonio mirror A portion of the image transmitted through the gonioscope 120 may be mirrored. Accordingly, portions of the images captured by the surgical microscope 108 that were received through the gonioscope 120 may be flipped to correspond to the actual orientation of the eye 102 such as using the approach described in U.S. Pat. No. 10,201,270, which is hereby incorporated herein by reference in its entirety.

Referring to FIG. 1D, in some embodiments, one or both of the imaging device 112 and the accessory device 114 may be mounted to the surgical microscope by an adjustable support 122. The adjustable support 122 may facilitate adjustment of the position of one or both of the imaging device 112 and accessory device 114 substantially (e.g., within 2 degrees of) parallel to the optical axis of the surgical microscope 108. The adjustable support 122 may also be adjustable in one or more other dimensions perpendicular to the optical axis of the surgical microscope 108. Controls for adjusting the position of the imaging device 112 and/or accessory device 114 may be manual or actuated. When actuated, an interface for adjusting the adjustable support 122 may include physical buttons mounted to the surgical microscope 108, voice commands, gesture controls, a touch screen, or other interface.

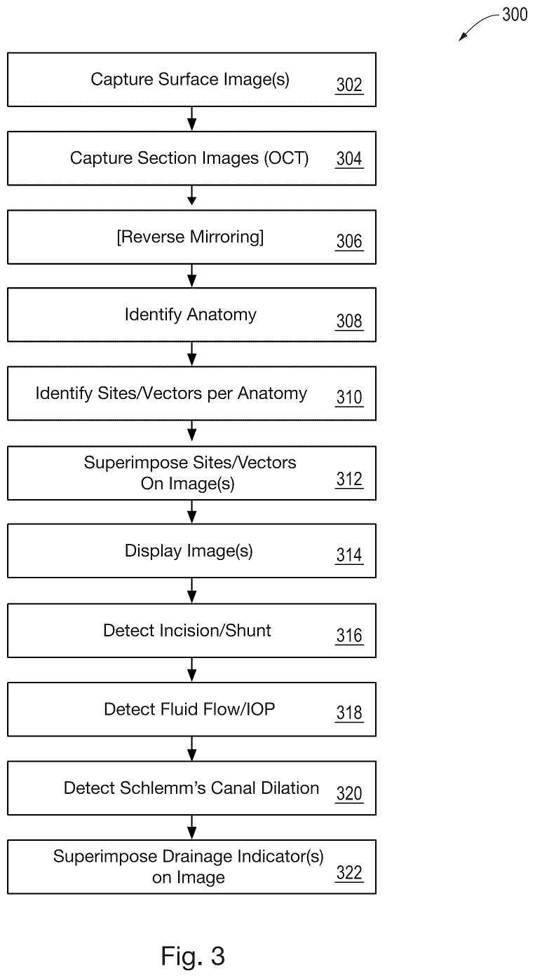

FIGS. 2 and 3 illustrate use of the operating environment 100 for the treatment of glaucoma. Referring specifically to FIG. 2 treatments for glaucoma often take place in the anterior segment 200 of the eye 102, which is located behind the transparent and spherical cornea 202 through which light enters the eye 102. The iris 204 is a ring of muscles defining the pupil of the eye through which light passes. The crystalline lens 206 is located behind the pupil and, together with the cornea 202, focuses light onto the light sensitive cells of the retina 208. The retina 208 is formed on the interior of the globe 210 of the eye opposite the anterior segment 200. The globe 210 of the eye between the lens 206 and the retina 208 is occupied by a transparent gel known as the vitreous 212.

The ciliary body 214 includes ligaments and muscles that connect the iris 204 and lens 206 to the choroid 216 of the eye. The muscles of the ciliary body 214 are responsible for altering the shape of the lens 206. The choroid 216 is a vascularized layer lining the globe 210 of the eye.

The ciliary body 214 produces the aqueous humor, which is the fluid that occupies the anterior segment 200. The aqueous humor washes over the lens 206 and iris 204 and flows to the perimeter of the anterior segment 200. The perimeter of the anterior segment includes structures that, when functioning normally, allow the aqueous humor to drain. These structures include the trabecular meshwork 218 and Schlemm's canal 220. The trabecular meshwork 218 seems to act as a filter, limiting the outflow of aqueous humor and providing a back pressure that directly relates to IOP. Schlemm's canal 220 is located beyond the trabecular meshwork 218. Schlemm's canal 220 is fluidically coupled to collector channels (not shown) allowing aqueous humor to flow out of the anterior segment 200.

Glaucoma may be treated by inserting the illustrated rod 222 into the anterior segment 200, such as through an incision in the limbus 224 at the boundary between the cornea 202 and the sclera (white) of the eye. The rod 222 is then used to place an incision and possibly a stent in one or more structures at the perimeter of the anterior segment 200 to facilitate drainage of the aqueous humor. For example, an incision or stent may be placed in the trabecular meshwork 218 to facilitate drainage into Schlemm's canal 220. In other approaches, a stent extends from the anterior segment into a suprachoroidal space between the choroid 216 and globe 210 of the eye.

Referring specifically to FIG. 3, the illustrated method 300 may be implemented using the operating environment 100 in order to facilitate performance of a glaucoma treatment. The method 300 may be executed by a computing device receiving images from the surgical microscope 108 and the imaging device 112 and any outputs of the accessory device 114 when the accessory device 114 is present and used.

The method 300 includes capturing, at step 302, one or more surface image of the eye 102 and capturing, at step 304, one or more section images of the eye 102. As used herein, “surface image” refers to images capturing light reflected from a surface of the eye 102 and/or transmitted and reflected by way of one or more transparent structures of the eye including the cornea 202 and lens 206. A surface image may be a visible light image, multi- or hyperspectral image, infrared image, or other type of image. A surface image may be one of two or more images providing a stereoscopic view of the eye 102. As used herein, “section image” refers to an image including a cross-section of tissues of the eye 102, including tissues at a depth that is not visible in surface images. In a section image, the depth within the tissue of the eye represented by a pixel of the image is known, whereas a surface image may flatten light reflected from various depths within the tissue of the eye 102 into a single image. A section image may be composed of a plurality of cross-sectional images forming a three-dimensional image. A section image may be a three-dimensional image that may be viewed along various section planes. In some embodiments, a section image is an OCT image. Section images captured at step 304 may compose a three-dimensional image of at least a portion of the eye 102, such as the anterior segment 200. The surface image and the section images may be registered with respect to one another, i.e., pixels representing anatomy in the surface image may be mapped to pixels (or voxels) of the three-dimensional image corresponding to the same anatomy.

Where a gonioscope 120 is used, the method 300 may include reversing, at step 306, an image received through the gonioscope 120 to undo mirroring imposed by the gonioscope 120, such as an image received from the surgical microscope 108. Where a gonioscope 120 is not used, step 306 may be omitted.

The method 300 includes identifying, at step 308, anatomy within the three-dimensional image and the one or more surface images. Identifying anatomy may include processing one or both of the three-dimensional image and the one or more surface images using a machine learning model. For example, for each item of anatomy of the eye to be identified, training data entries may be created that include a three-dimensional image and one or more surface images and labels indicating portions of the three-dimensional image and one or more surface images corresponding to the item of anatomy. The training data entries may then be used to train a machine learning model to identify that item of anatomy at step 308. There may be multiple machine learning models each trained to identify one or more different items of anatomy.

The method 300 may include identifying, at step 310, one or more sites at which to place an incision or stent according to the anatomy. For example, the sites may be selected to place an incision or stent passing into Schlemm's canal 220. Accordingly, the sites select at step 310 may be positioned on the trabecular meshwork 218 over Schlemm's canal 220. Drainage channels conduct fluid away from the Schlemm's canal. Accordingly, the insertion sites may also be selected to be adjacent, e.g., within 0.5 mm of the drainage channels. Identifying the one or more sites may include identifying a sites having a number and distribution, e.g., minimum separation between them, specified in a treatment plan. Sites may be identified on the trabecular meshwork 218, a bleb, or elsewhere on the eye 102. Step 310 may further include identifying a vector for each insertion site. The vector may specify the direction along which an incision should be made or a stent should be made inserted at a site, such as in order to extend into Schlemm's canal or have a desired relationship with respect to other anatomy of the eye 102.

The method may include superimposing, at step 312, one or more representations of the one or more sites on an image, such as the surface image from step 302, a section plane from step 304, a rending of the three-dimensional image, or some other image. Step 312 may further include superimposing a representation of each vector identified at step 310 on the image. The image with the superimposed representations of the sites and or vectors may then be displayed at step 314 on the display device 118, a display (e.g., a stereoscopic display) of the surgical microscope 108, or elsewhere.

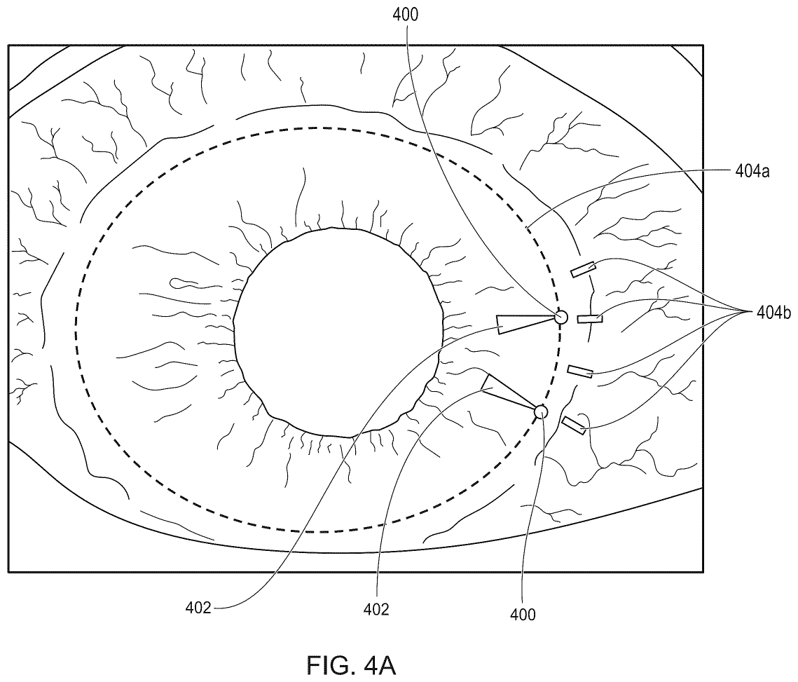

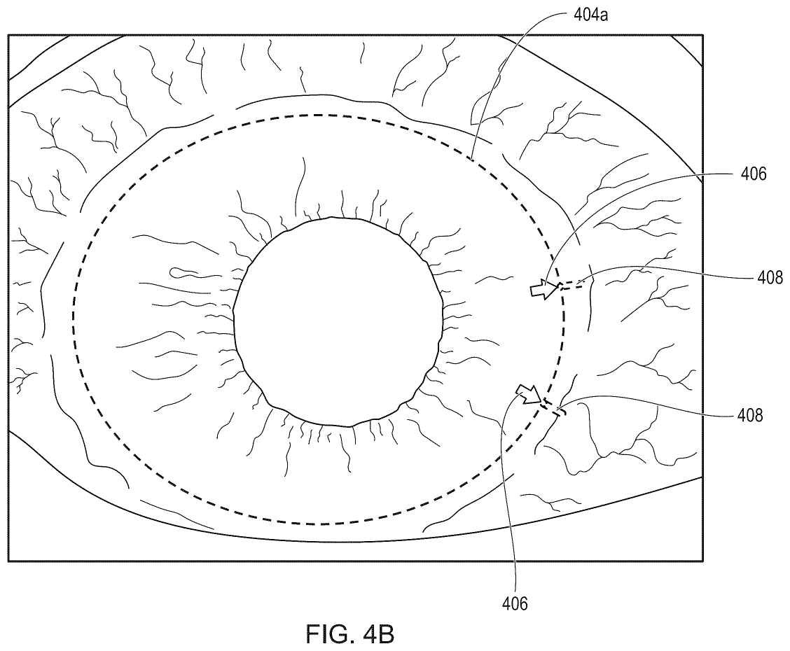

For example, referring to FIG. 4A, the illustrated image may be marked with site markers 400 representing the sites identified at step 310 and vectors markers 402 representing the vectors identified at step 310. Other items of anatomy may be marked, such a marker 404a labeling the pigmented trabecular meshwork, which is often the selected location for placing an incision or stent. One or more markers 404b may label the location of drainage canals.

Referring again to FIG. 3, the method 300 may further include detecting, at step 316, the creation of an incision or placement of a stent. Step 316 may include detecting movements of the rod 222, detecting changes to the trabecular meshwork in a three-dimensional image and/or surface image captured after creation of the incision or placement of the stent, detecting a marker or otherwise detecting the stent in the three-dimensional image, or some other approach.

The method 300 may include detecting, at step 318, fluid flow through the incision or stent. Step 318 may additionally or alternatively include detecting IOP of the eye 102. Detecting fluid flow may be detected using the accessory imaging device 112. For example, velocity of flow of fluid through the incision and/or stent may be obtained by detecting a red/blue shift in reflected light using any approach known in the art. The velocity of fluid flow may be measured in specific areas, such as in the region of the incision or stent. Dye may be injected into the anterior segment to facilitate visualization of fluid flow. Fluid flow may be inferred by detecting a change or rate of change in IOP sensed after creation of the incision, such as where the accessory device 114 is an IOP sensor.

The method 300 may include detecting, at step 320, a degree of dilation of Schlemm's canal 220. For example, Schlemm's canal 220 may be identified in a first three-dimensional captured prior to creation of an incision and/or placement of a stent. Schlemm's canal 220 may then be identified in one or more second three-dimensional images captured after creation of the incision and/or placement of the stent. The size of the representations of the Schlemm's canal 220 in the first three-dimensional image and the one or more second three-dimensional images may then be calculated, such as the number of voxels identified as being part of a representation of the Schlemm's canal. A degree of dilation may therefore be calculated as a ratio of the number of voxels representing the Schlemm's canal 220 in the first three-dimensional image and the number of voxels representing the Schlemm's canal 220 in one of the second three-dimensional image.

The method 300 may include superimposing, at step 322, an indicator of drainage on an image of the eye 102, such as a surface image of the eye 102 captured before or after creation of the incision or placement of the stent. For example, as shown in FIG. 4B, the indicator of drainage may include a symbol, such as the illustrated arrow 406. Attributes of the symbol may indicate the degree of drainage, such as size (e.g., larger indicating greater drainage) color (green=adequate improvement to drainage, yellow=insufficient improvement to drainage, red=no significant improvement to drainage). The attributes of the indicator of drainage may be a function of some or all of the amount of fluid flow detected at step 318 and the degree of dilation of Schlemm's canal 220 detected at step 320. Step 322 may additionally include superimposing representations 408 of the incision and/or stent on the image at locations corresponding to those detected at step 316 either with or without displaying site markers showing the intended location for placement of the incision and/or stent.

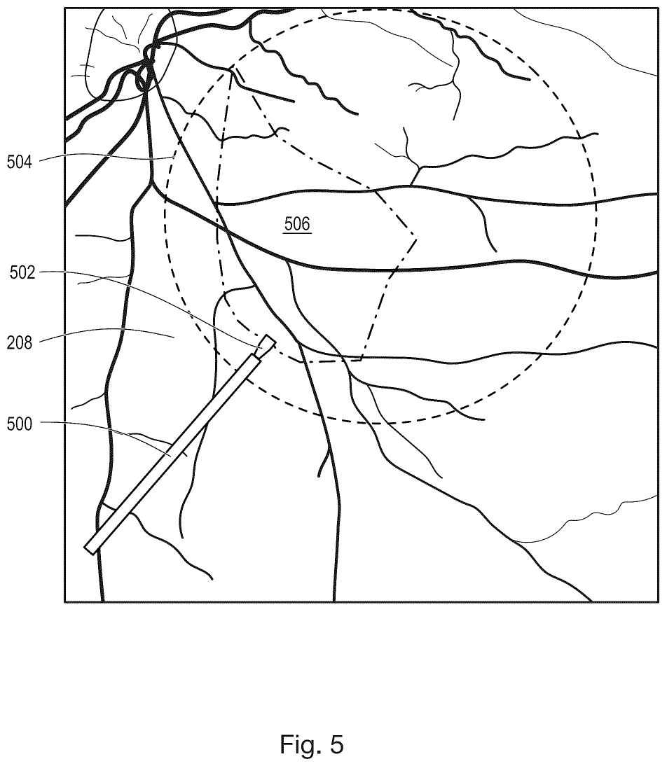

Referring to FIG. 5, the operating environment 100 may be used to facilitate peeling of a membrane from the retina 208, such as an interior limiting membrane (ILM) or epiretinal membrane (ERM). The membrane may be peeled using an instrument 500 inserted into the eye and having forceps 502 that may be extended from the instrument 500 and actuated to grasp the membrane. A peeling treatment may include peeling the membrane within a boundary 504, such as over the macula of the eye 102. A peeling treatment may include peeling all of the membrane within the boundary 504 in a single step or may be performed in pieces. For example, a peeled area 506 may be peeled in a first grasping and peeling step and the remainder being peeled in one or more other grasping and peeling steps.

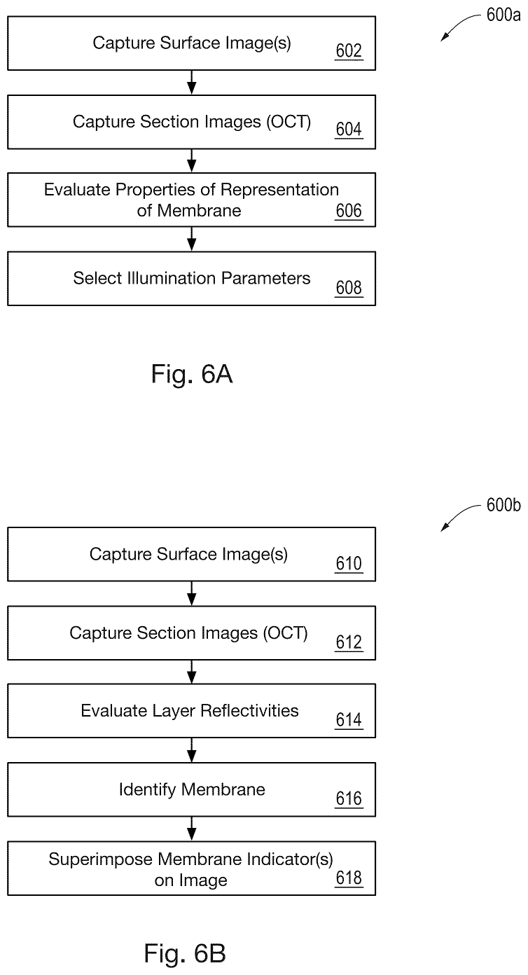

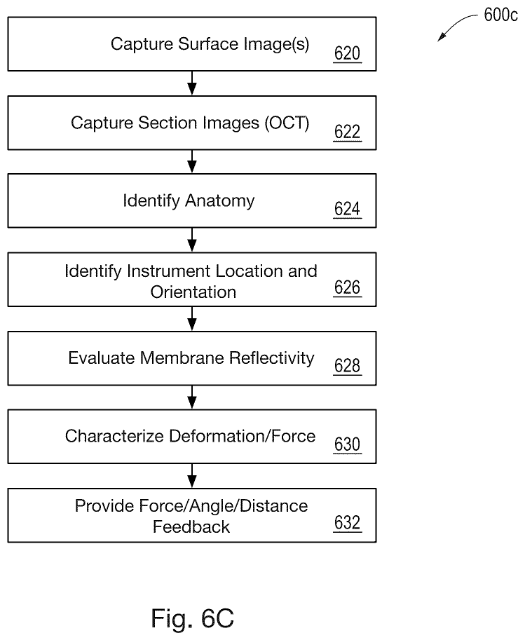

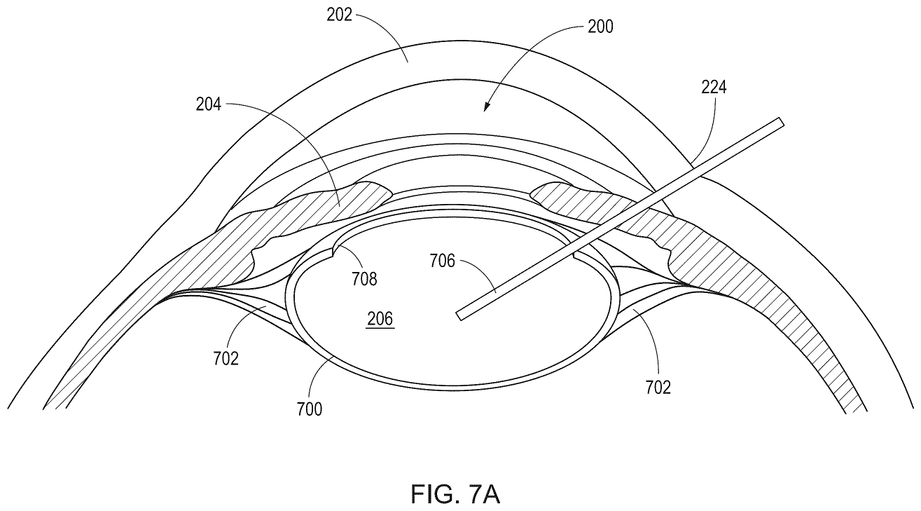

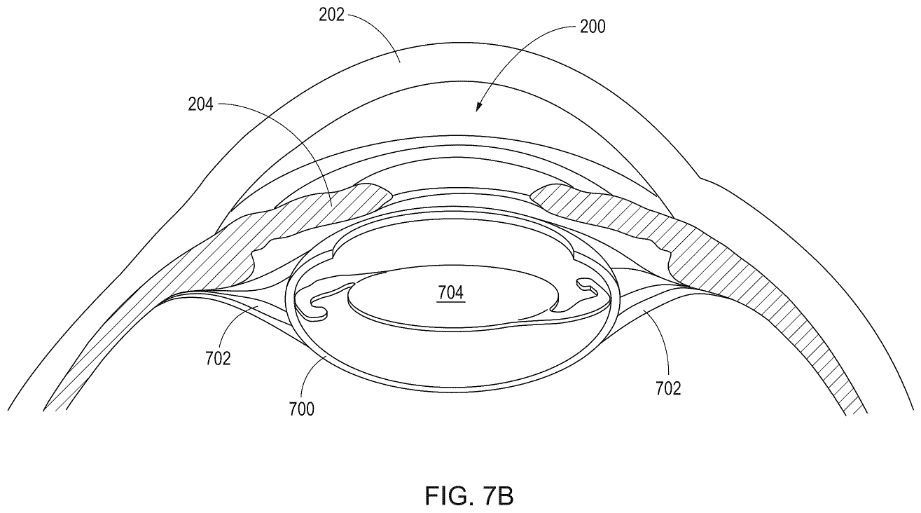

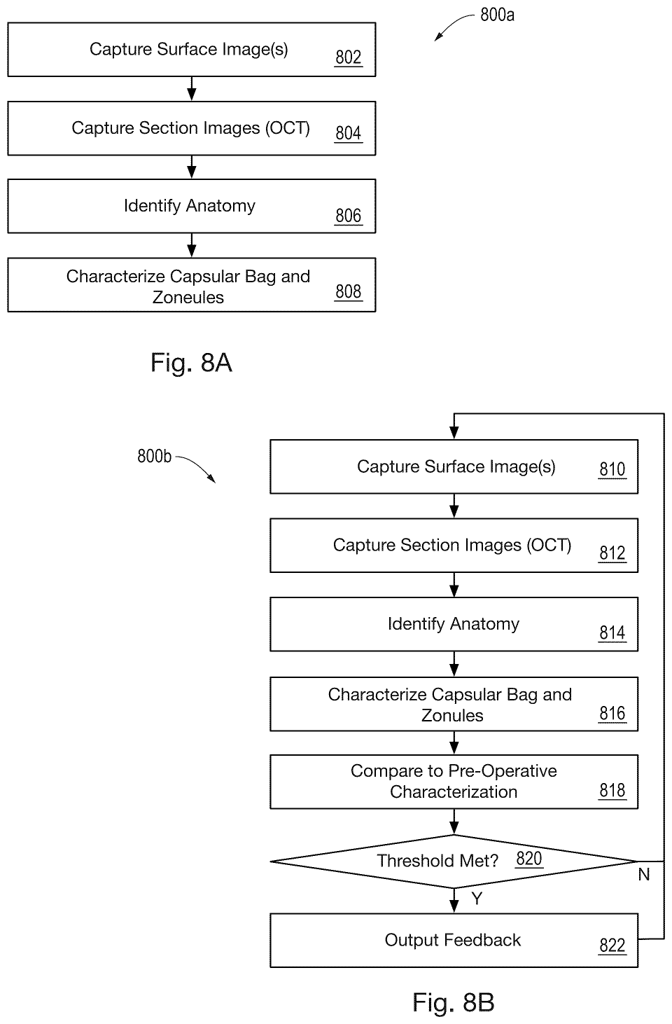

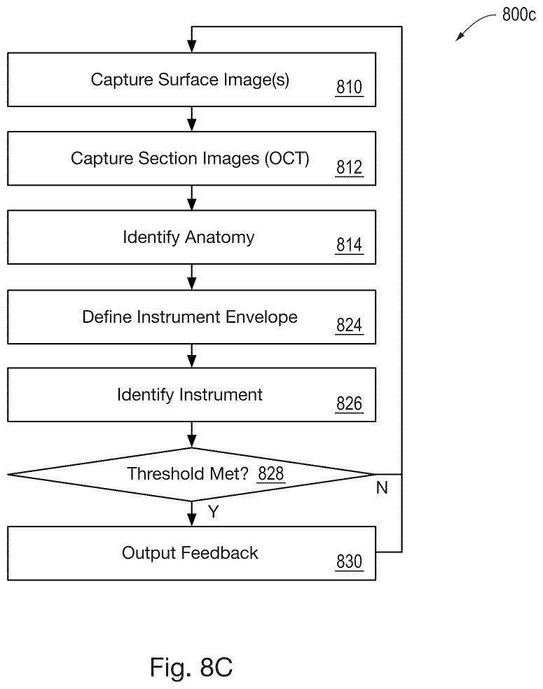

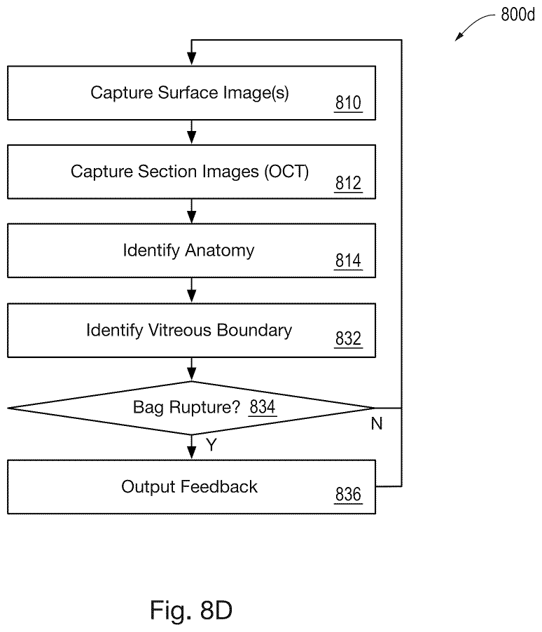

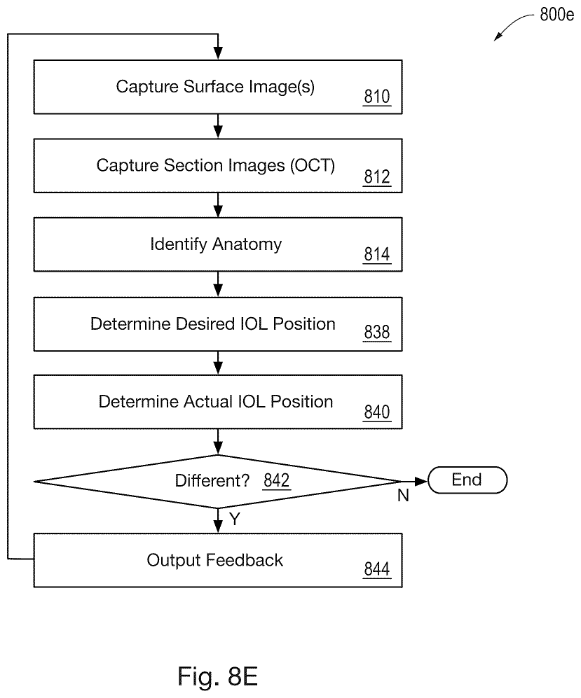

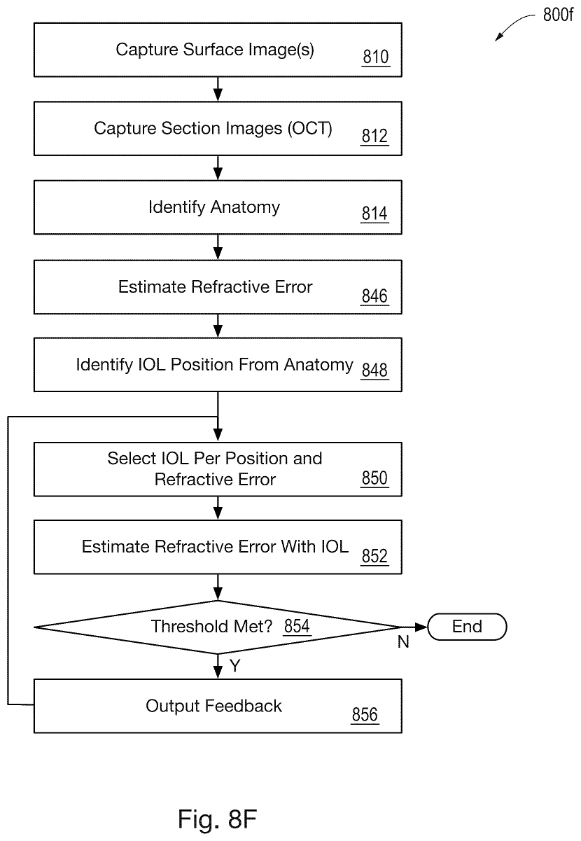

FIGS. 6A, 6B, and 6C are methods that may be performed using the operating environment 100 to facilitate a peeling treatment. When performing a peeling treatment, the accessory device 114 may be embodied as a light source. The light source may be controllable with respect to a plurality of parameters, such as color and intensity. The light source may be a multi- or hyperspectral light source such that the plurality of parameters include the intensity of light within each of three, four, five, or more wavelength bands.

Referring specifically to FIG. 6A, a method 600a may include capturing, at step 602, one or more surface images of the retina 208 and capturing, at step 604, one or more section images of the retina 208. The one or more surface images may be captured with the retina 208 being illuminated by a light source of the surgical microscope 108 alone or with the accessory device 114 providing light according to initial values for the plurality of parameters.

The method 600a may include evaluating, at step 606, properties of a representation of the membrane in one or both of the one or more surface images and the one or more section images. The properties of the representation of the membrane may include image quality metrics that correspond to the surgeon's ability to clearly see the membrane during the peeling operation. Image quality metrics may include values such as sharpness, contrast, saturation, or other metrics of image quality. The properties of the representation of the membrane may be the output of a machine learning model. For example, each training data entry of a plurality of training data entry may include one or more images captured during a previous peeling procedure as an input and one or more human-assigned metrics of the quality of the representation of the membrane in the one or more images. A machine learning model may then be trained with the plurality of training data entries to output, for a given input image, one or more metrics of image quality of the representation of a membrane in the input image. Alternatively, a machine vision algorithm may be configured to similarly output one or more metrics of image quality.

The method 600a may include select, at step 608, values for the plurality of parameters based on the evaluating of step 606. The values for the plurality of parameters may be selected based on the one or more metrics of image quality obtained at step 606. The values for the plurality of parameters may include applying a predefined algorithm that translates the one or more metrics into corresponding values for the plurality of parameters, the predefined algorithm configured to select values for the plurality of parameters that will improve the one or more metrics, i.e., cause subsequent surface images to enable better visualization of the retina 208 and the membrane to be removed. Alternatively or additionally, step 608 may include a search algorithm in which the retina 208 is illuminated with light generated according to a set of value for the plurality of parameters, a surface image of the retina 208 is captured, and the one or more quality metrics are calculated for the surface image. Multiple sets of values in a search space may be tested in this manner and the set of values achieving the best one or more metrics of image quality may then be selected.

FIG. 6B illustrates a method 600b for identifying a region of the retina 208 covered by a membrane. The method 600b is particularly suitable for identifying pathological membranes, such as an ELM, which may have irregular shapes and locations. The area of the ILM to be removed from the retina 208 may be easily identified based on anatomy of the retina, i.e., a circle of a known radius centered on the fovea of the retina 208, which is identifiable due to higher pigmentation and lack of vascularization. However, in some embodiments, the method 600b may also be used to identify the ILM.

The method 600b may include capturing one or more surface images and one or more section images at steps 610 and 612 as described above. The method 600b includes evaluating at step 614, reflectivities of different areas of the retina in the one or more surface images and possibly the one or more section images. Step 614 may include evaluating variation in reflectivities within individual wavelength bands.

The method 600b may include identifying, at step 616, a representation of the membrane is identified in the one or more surface images and possibly the one or more section images. Step 616 may include identifying the membrane based on changes in the reflectivities evaluated at step 614, e.g., changes in reflectivities indicating the boundary of the membrane.

The method 600b may include superimposing, at step 618, a membrane indicator on an image, such as one or more of the surface images. For example, as shown in FIG. 5, a boundary 504 of the membrane may be shown as a line, shaded region, or other visual indicator. The method 600b may be repeated during a peeling procedure such that a peeled area 506 that is free of the membrane is also represented in the image, such as by lacking the membrane indicator or being outside of a line indicating the current boundary 504 of the membrane. In some embodiments, a numerical, textual, or other indicator indicates the amount of the membrane that has been peeled and/or remains to be peeled. The image with the membrane indicator superimposed thereon may be displayed to a surgeon on the display device 118, on an internal display of the surgical microscope 108, or other display device.

Referring to FIG. 6C, the operating environment 100 may be used to perform the illustrated method 600c in order to provide feedback during a peeling treatment. The method 600c may include capturing one or more surface images at step 620, capturing one or more section images at step 622, and identifying anatomy at step 624. Steps 620, 622, 624 may be performed as for any of the methods described hereinabove.

The method 600c may further include identifying, at step 626, the location and possibly orientation of a surgical instrument, such the instrument 500 and forceps 502, in one or both of the one or more surface images and the one or more section images. For example, the position and orientation of the surgical instrument may be determined in three dimensions from a three-dimensional image formed by the one or more section images.

The method 600c may further include evaluating, at step 628, membrane reflectivity. In particular, a surface image, and within the portion of the surface image corresponding to the membrane, variation in reflectivity of the membrane may be evaluated. For example, variation in reflectivity in a region around the location of the forceps 502. Variation in reflectivity occurs when the membrane is deformed. Accordingly, the variation in reflectivity may be used to characterize deformation of the membrane at step 630. Step 630 may include applying a predefined function, algorithm, or machine learning model to translate the variation in reflectivity to characterization of deformation. The deformation may be translated to a force exerted by the forceps 502 or the reflectivity may be directly translated to an estimate of force exerted by the forceps 502.

The method 600c may include providing, at step 632, feedback to a surgeon regarding the force exerted by the surgeon on the retina 208. For example, feedback may be a color coded indicator output to the display device 118, an internal display of the surgical microscope, an audible signal, haptic feedback, or other feedback. For example, visual feedback may be a red symbol or overlay where force is excessive, and a green symbol or overlay where force is in a range appropriate for membrane grasping, and a yellow symbol or overlay where the force is too low for membrane grasping.

Other forms of feedback may also be provided. For example, the orientation of the surgical instrument relative to the retina 208 may be compared to a range of acceptable relative orientations for grasping the membrane and feedback provided accordingly. Feedback may be a visual, audible, or textual message indicating a needed change in orientation. Feedback may be in the form of an overlay superimposed on a surface image or a rendering based on the three-dimensional image indicating the correct orientation of the surgical instrument.

In some embodiments, feedback is distance feedback. For example, it may be undesirable for the forceps 502 to contact anatomy that is not to be peeled, such as areas of the retina 208 not covered by the membrane to be peeled or other anatomy of the eye 102. Accordingly, if the location of the forceps 502 is within a threshold distance of anatomy that is not to be peeled, feedback may be provided. Feedback may be a visual, audible, or textual message indicating instructing the surgeon to stop moving the forceps 502 along a current trajectory. Distance feedback may be a displayed numerical or other indicator, e.g., a distance in microns or other units, indicating the distance of the forceps from the retina 208, which may include the distance to the region of the retina 208 to be peeled to help the surgeon when bringing the forceps into contact with the membrane.

Referring to FIGS. 7A and 7B, during a surgery for cataracts, the lens 206 is removed through phacoemulsification. The lens 206 is located within the capsular bag 700, which is connected to the ciliary body 214 by filaments known as zonules 702. An intraocular lens (IOL) 704 is then placed within the capsular bag 700 to replace the lens 206. The success of cataract surgery depends on the condition of the capsular bag 700 and zonules 702. If the capsular bag 700 ruptures or the zonules 702 break, a different type of IOL and different placement location will be needed. The success of a cataract surgery also depends on the correct selection of the IOL 704 and placement of the IOL 704 relative to the retina 208 and cornea 202 to reduce the postoperative refractive error of the eye 102.

Cataract surgery is performed by inserting an instrument 706 through an incision, typically located at the limbus 224. The instrument 706 is used to create an opening 708 (rhexis) in the capsular bag 700 through which the lens 206 is removed and the IOL 704 is inserted.

FIGS. 8A to 8F illustrate methods that may be performed using the operating environment 100 in order to facilitate cataract surgery. FIG. 8A illustrates a method 800a that may be performed preoperatively. The method 800a may include some or all of capturing, at step 802, one or more surface images of the eye 102 and capturing, at step 804, one or more section images of the eye 102, particularly of the lens 206, ciliary body 214, capsular bag 700, and zonules 702. Since the method 800a is performed preoperatively, the one or more surface images and one or more section images may be obtained using imaging devices that are not mounted to otherwise associated with a surgical microscope 108.

The method 800a may include identifying, at step 806, anatomy represented in the one or more section images and one or more surface images. In particular, representations of the lens 206, ciliary body 214, capsular bag 700, and zonules 702 may be identified according to any of the approaches described hereinabove. The capsular bag 700 and zonules 702 may then be characterized at step 808. The characterization of the capsular bag 700 may include an average thickness of the capsular bag 700, a minimum thickness of the capsular bag 700, locations of a regions of the capsular bag 700 below a thickness threshold, or other characterizations. The characterization of the zonules 702 may include a number or average density of the zonules (e.g., per unit area of the surface of the capsular bag), average diameter of the zonules 702 (average diameter at the thinnest point for all zonules 702), minimum diameter of the zonules 702, or other characterization.

Referring to FIG. 8B, the method 800b may be executed by the operating environment 100 using preoperative characterizations of the capsular bag 700 and zonules 702 according to the method 800a. The method 800b includes some or all of capturing, at step 810, one or more surface images of the eye 102, capturing, at step 812, one or more section images of the eye 102, and identifying, at step 814, anatomy of the eye. Steps 810, 812, 814 may be performed according to any of the approaches described hereinabove.

The method 800b includes characterizing, at step 816, one or both of the capsular bag 700 and zonules 702 according to one or both of the one or more surface images and one or more section images. Step 816 may be performed in the same manner as step 808 described above. The method 800b may include comparing, at step 818, the characterization of the capsular bag 700 and zonules 702. To the pre-operative characterization of the capsular bag 700 and zonules 702 according to the method 800a.

If one or more differences between the characterizations of step 818 and the pre-operative characterization are found, at step 820, to exceed corresponding thresholds, the method may include outputting, at step 822, feedback to the surgeon. The feedback may be visual or textual information communicating differences between the pre-operative characterizations and the characterizations of step 818. For example, the feedback may superimpose markings on a surface image or a rendering from a three-dimensional image, the markings showing areas of the capsular bag 700 that are thinner or looser than indicated in the preoperative data or zonules 702 that are thinner, looser, or missing relative to the preoperative characterizations. The surgeon may therefore determine whether a proposed cataract treatment is still feasible or whether changes should be made. For example, whether IOP should be reduced to reduce pressure on the capsular bag 700.

FIG. 8C illustrates a method 800c for providing feedback to a surgeon to help avoid rupture of the capsular bag 700 during cataract surgery. The method 800c may include some or all of capturing, at step 810, one or more surface images, capturing, at step 812, one or more section images, and identifying, at step 814, anatomy as described above.

The method 800c may include defining, at step 824, an instrument envelope. The instrument envelope may include, a circular path for performing rhexis, i.e., cutting an opening in the capsular bag 700 through which the lens 206 can removed. The circular path may be defined with respect to the detected inner surface of the iris 204, such as a path that is offset inwardly from the iris 204 by a predefined margin. The instrument envelope may be defined with respect to the interior of the capsular bag 700 for phacoemulsification. For example, the instrument envelope may include a volume within the capsular bag 700, such as a volume offset from the inner surface of the capsular bag by some margin, such as between 0.01 and 0.1 mm.

The method 800c may include identifying, at step 826, a representation of an instrument, such as a phaco-vit tool, in a three-dimensional image composed of the one or more section images. If the instrument, such as a distal end thereof, is found, at step 828, to be within a threshold distance of the instrument envelope, the method 800c may include outputting, at step 830, feedback to the surgeon. The feedback may indicate simply that a potential collision on the instrument envelope. The feedback may also indicate the direction in which to move the instrument to avoid collision with the instrument envelope. The feedback may include a visual alert output on the display device 118 or a display device internal to the surgical microscope 108. The alert may be an audible alert output through a speaker. The alert may be haptic feedback output through a haptic device mounted on or within a handpiece to which the instrument is mounted. The method 800c may be repeated throughout phacoemulsification as shown in FIG. 8C.

FIG. 8D illustrates a method 800d that may be performed using the operating environment 100 to facilitate the early detection of capsular bag rupture. With the lens 206 removed, the capsular bag 700 is maintains the vitreous 212 in the posterior segment. If the capsular bag 700 ruptures, the vitreous 212 may begin to leak out of the posterior segment. However, because the vitreous 212 and the capsular bag 700 are transparent, it may be difficult for the surgeon to detect bag rupture. Using the operating environment 100 it is possible to detect the different indices of refraction of the vitreous 212, capsular bag 700, and an infusion fluid used to fill the posterior segment during phacoemulsification.

The method 800d may include some or all of capturing, at step 810, one or more surface images, capturing, at step 812, one or more section images, and identifying, at step 814, anatomy as described above.

The method 800d may include identifying, at step 832, the vitreous boundary. Step 832 may include detecting a blob of pixels (or voxels) within the posterior segment and detecting the boundary of this blob. For example, assuming the center of the posterior segment (or some point closer to the retina 208) is definitely occupied by vitreous, step 832 may include working outwardly from such a point to detect boundaries at which the index of refraction changes or some other anatomical boundary, such as the retina and/or choroid are reached.

The method 800d may include evaluating, at step 834, whether bag rupture has occurred. For example, if the representation of the vitreous identified at step 832 is found to extend into the capsular bag, past the iris, or elsewhere in the anterior segment 200, the capsular bag may be found to have ruptured. If so, then feedback may be output at step 836. The feedback may be in the form of a visual message, such as text or other symbol, output to the display device 118 or a display device internal to the surgical microscope 108. Feedback may also be in the form of an audible message output by a speaker, haptic feedback through a handpiece, or other type of feedback.

FIG. 8E illustrates a method 800e that may be performed using the operating environment 100 to facilitate the placement of an IOL 704. With the lens 206 removed, the IOL 704 may be placed within the capsular bag 700. Correct placement of the IOL may be facilitated using the operating environment 100 in order to avoid rupture of the capsular bag 700 and reduce post-operative refractive error.

The method 800e may include some or all of capturing, at step 810, one or more surface images, capturing, at step 812, one or more section images, and identifying, at step 814, anatomy as described above.

The method 800d may include determining, at step 838, a desired IOL position relative to the anatomy identified at step 814. For example, a treatment plan for a cataract surgery may specify a desired position (e.g., along the optical axis of the eye 102) and possibly orientation (e.g., angular position about the optical axis of the eye 102) for the IOL 704. The desired position may be defined with respect to anatomy, such as the capsular bag 700, iris 204, ciliary body 214, or other item of anatomy. Determining the desired IOL position may therefore include identifying a position of the IOL with respect to anatomy identified at step 814 corresponding the relative position of the IOL with respect to the anatomy in the treatment plan.

The method 800d may include determining, at step 840, an actual location of the IOL. Step 840 may include identifying voxels corresponding to the IOL in the three-dimensional image composed of the one or more section images. The method 800d may include determining, at step 842, whether a difference between the actual IOL position and the desired IOL position is different by more than a threshold amount, such as more than a first threshold distance along the optical axis, more than a first threshold angle about the optical axis, and/or more than a second threshold angle in a plane parallel to the optical axis, i.e., tilt.

If the difference between the actual IOL position and the desired IOL position is found to exceed one or more thresholds, the method 800e may include outputting, at step 844, feedback to the surgeon. Feedback may be in the form of a text or audible output communicating translation or rotation of the IOL required to achieve the desired IOL position. Feedback may be in the form of an overlay superimposed on a surface image or a rendering of the three-dimensional image, the overlay showing the desired IOL position. The overlay may further highlight the representation of the IOL 704 in order to more clearly show the difference between the actual and desired IOL positions.

FIG. 8F illustrates a method 800f for selecting an IOL 704, i.e., the dioptric power or other properties of an IOL 704. The method 800f includes some or all of capturing, at step 810, one or more surface images of the eye 102, capturing, at step 812, one or more section images of the eye 102, and identifying, at step 814, anatomy of the eye 102. Steps 810, 812, 814 may be performed according to any of the approaches described hereinabove.

The method 800f may include estimating, at step 846, the refractive error of the eye 102. Estimating the refractive error may include performing ray tracing or other algorithm to estimate the focal point of the eye by taking into account refraction of the cornea 202, lens 206, capsular bag 700, aqueous humor (fluid filling the anterior segment 200), and vitreous 212.

The method 800f may further include identifying, at step 848, a position of an IOL according to the anatomy identified at step 814. For example, based on the dimensions of the capsular bag 700 and known dimensions of an IOL, the location at which the IOL will seat within the capsular bag 700 may be identified. The IOL may be selected from a collection of available IOLs to seat within the volume offered by the capsular bag 700.

The method 800f includes selecting, at step 850, a dioptric power for an IOL positioned at the position selected at step 848 and based on the refractive error calculated at step 846. The dioptric power may be the power of a refractive component of a multi-focal IOL. The method 800f may include estimating, at step 852, refractive error of the IOL with the selected dioptric power when placed at the position identified at step 848. Step 852 may include performing ray tracing or other modeling technique to estimate the position of the focal point of the combined eye 102 and IOL. If the estimated refractive error of step 852 is found, at step 854, to meet a threshold, the method 800f. Otherwise, the method 800f may continue at step 850. The method 800f may further include outputting, at step 856, feedback, such as report of the refractive error estimated at step 852.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine coupled to components of the operating environment 100. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A system for performing ophthalmic treatments, the system comprising:
a surgical microscope configured to capture surface images of an eye of a patient;
an imaging device mounted to the surgical microscope and configured to capture section images of the eye of the patient; and
a controller coupled to the surgical microscope and the imaging device, the controller configured to receive the surface images and section images and provide feedback facilitating performance of the ophthalmic treatments based on the section images and the surface images.

2. The system of claim 1, wherein the imaging device is an optical coherence tomography (OCT) imaging device.

3. The system of claim 1, wherein the imaging device is mounted to the surgical microscope by an adjustable support.

4. The system of claim 1, wherein the imaging device is mounted to a ring secured to the surgical microscope.

5. The system of claim 1, further comprising an accessory device secured to the surgical microscope, the accessory device including at least one of a sensor or a light source.

6. The system of claim 5, wherein the accessory device is an intraocular pressure sensor.

7. The system of claim 1, wherein the ophthalmic treatments include a glaucoma treatment and the feedback includes a feedback image derived from at least one of the surface images and the section images, the feedback image including markings showing sites for at least one of making an incision or placing a stent.

8. The system of claim 1, wherein the ophthalmic treatments include a glaucoma treatment and the feedback includes a feedback image derived from at least one of the surface images and the section images, the feedback image including markings representing drainage from an anterior segment of the eye of the patient.

9. The system of claim 1, wherein the ophthalmic treatments include a retinal membrane peeling treatment and the feedback includes a feedback image derived from at least one of the surface images and the section images, the feedback image including superimposed markings superimposed on a representation of a retinal membrane in the feedback image.

10. The system of claim 1, wherein the ophthalmic treatments include a retinal membrane peeling treatment and the feedback includes feedback regarding angle of a surgical instrument.

11. The system of claim 1, wherein the ophthalmic treatments include a retinal membrane peeling treatment and the feedback includes feedback regarding exerted force of a surgical instrument.

12. The system of claim 11, wherein the controller is configured to estimate the exerted force based on reflectivity of a retinal membrane.

13. The system of claim 1, wherein the ophthalmic treatments include phacoemulsification and the feedback includes feedback regarding a condition of a capsular bag and zonules of the eye of the patient.

14. The system of claim 1, wherein the ophthalmic treatments include placement of an intraocular lens (IOL) and the feedback includes feedback regarding a position of the IOL.

15. The system of claim 1, wherein the controller is configured to detect representations of an instrument in one or both of the surface images and the section images, the controller being further configured to output feedback regarding proximity of the instrument to anatomy of the eye of the patient.

16. A method for performing an ophthalmic treatment the method comprising:
capturing, by a surgical microscope configured to capture surface images of an eye of a patient;
capturing, by an imaging device mounted to the surgical microscope, section images of the eye of the patient;
receiving, by a controller coupled to the surgical microscope and the imaging device, the surface images and section images; and
providing, by the controller, feedback facilitating performance of the ophthalmic treatment according to the surface images and the section images.

17. The method of claim 16, wherein the imaging device is an optical coherence tomography (OCT) imaging device.

18. The method of claim 16, wherein the ophthalmic treatment is a glaucoma treatment, the method further comprising:
generating, by the controller, a first feedback image derived from at least one of the surface images and the section images, the first feedback image including markings showing sites for at least one of making an incision or placing a stent;
outputting, by the controller, the first feedback image to a display device;
estimating, by the controller, drainage from an anterior segment of the eye of the patient based on the section images;
generating, by the controller, a second feedback image derived from at least one of the surface images and the section images, the second feedback image including markings representing drainage from the anterior segment of the eye of the patient; and
outputting, by the controller, the second feedback image to the display device.

19. The method of claim 16, wherein the ophthalmic treatment is a retinal membrane peeling treatment, the method further comprising:
evaluating, by the controller, reflectivity of a retinal membrane;
calculating, by the controller, an estimated force exerted on the retinal membrane according to the reflectivity; and
outputting, by the controller, the feedback, the feedback based on the estimated force.

20. The method of claim 16, further comprising:
detecting, by the controller, representations of an instrument in one or both of the surface images and the section images; and
outputting, by the controller, the feedback, the feedback corresponding to proximity of the instrument to anatomy of the eye of the patient.
